(19) United States
(12) Patent Application Publication
(10) Pub. No.: US 2022/0257687 A1
(43) Pub. Date: Aug. 18, 2022

(54) MEMBRANE FILTRATION OF PLANT EXTRACTS BY MEANS OF CYCLODEXTRIN

(71) Applicant: Bionorica SE, Neumarkt (DE)

(72) Inventors: Moritz RUBNER, Pielenhofen (DE); Silvia FERSCH, Beilngries (DE)

(21) Appl. No.: 17/630,658

(22) PCT Filed: Jul. 31, 2020
(86) PCT No.: PCT/EP2020/071738

§ 371 Date: Jan. 27, 2022

(30) Foreign Application Priority Data
Jul. 31, 2019 (EP) 19189464.1

(51) Int. Cl. A61K 36/53; A61K 36/25; A61K 47/40; A23L 33/105; A23L 33/125; B01D 61/02

(52) U.S. Cl. CPC A61K 36/53; A61K 36/25; A61K 47/40; A23L 33/105; B01D 2311/08; B01D 61/025; A61K 2236/333; A61K 2236/53; A23V 2002/00; A23L 33/125

(57) ABSTRACT

The present invention relates to a method for producing an alcohol-reduced or alcohol-free composition of at least one alcohol-containing plant extract by means of membrane filtration using cyclodextrin.

### CROSS-REFERENCE TO RELATED APPLICATIONS

This application is a national stage application (under 35 U.S.C. § 371) of PCT/EP2020/071738, filed Jul. 31, 2020, which claims benefit of European Application No. 19189464.1, filed Jul. 31, 2019, both of which are incorporated herein by reference in their entirety.

The present invention relates to a method for producing an alcohol-reduced or alcohol-free composition of at least one alcohol-containing plant extract by means of membrane filtration using cyclodextrin.

Medicinal plants contain, possibly enriched in certain parts of plants such as roots, leaves, flowers or fruits, ingredients having a pharmacological effect and forming the basis for a considerable number of medicines and food supplements. There are various methods of obtaining these ingredients, most of which work on the principle of any type of extraction, including maceration or percolation of the plants using a suitable extractant or solvent, but mostly alcohol and, as a result of this, yield a more or less selective solution and enrichment of certain herbal active ingredients or groups of active ingredients in the extractant or extract. Plant extracts containing alcohol are therefore obtained. However, medicines, extracts and fluid extracts based on plant extracts that contain alcohol are constantly being criticized. Usually, aqueous-ethanolic solvents which can contain 30 to 70% ethanol are used as extraction agents for plant extracts. For example, fluid extracts are introduced in dosage forms such as drops and juices.

There is a great need to provide alcohol-free agents, in particular medicines made from plant extracts.

Membrane filtration is a pressure-operated filtration process for selective separating molecules having a molecular weight of at least 100 daltons up to a size of 5 μm, usually at low temperatures, for example, ambient temperature without phase change.

A particular embodiment of membrane filtration is reverse osmosis. Reverse osmosis is a high-pressure process used for separating water or alcohol from process fluids, in particular for concentrating low molecular weight compounds. Reverse osmosis is a physical process for concentrating substances dissolved in liquids, in which the natural osmosis process is reversed with pressure.

Further embodiments of membrane filtration depending on the molecular size and applied pressures include both ultrafiltration and nano- and microfiltration.

A food technology application of membrane filtration known in the prior art, in particular reverse osmosis, is the production of alcohol-free beer.

In the course of membrane filtration, a permeate (=filtrate) containing the removed alcohol and water is created from the fluid or feed used, while the retentate is retained on the membrane and consequently concentrated (concentrate).

Furthermore, EP 2621505 B1 describes the dealcoholization of plant extracts such as thyme, fennel, etc. using dispersants and wetting agents, in particular based on oils, fats and fatty acids, lecithins and glycerides.

However, there is a need to further improve the dealcoholization of plant extracts.

Cyclodextrins are widely described in the prior art, but the inventors were able to use cyclodextrins advantageously for the dealcoholization of plant extracts for the first time. In particular, the suitability of cyclodextrins for the production of inclusion compounds allows the molecule size to be increased, so that retention on the membrane can advantageously be achieved during membrane filtration. Such molecules cannot ultimately be preferably low molecular weight compounds, such as terpenes, monoterpenes, thymol, carvacrol, limonene, 1,8-cineol and 1,4-cineol, p-cymene, anethole, eugenol, fenchone, myristicin, vanillin, estragole, zingiberol, pinene, linalool, terpineol, myrcene, menthones, menthol, phellandren, menthane, carveol and dihydrocarveol, carvone, thujone, borneol, 3-carene, anethole, estragole and many others.

This further includes the respective stereoisomers, diastereomers, enantiomers such as (+), (−), alpha, beta, R and S etc.

The invention therefore relates to a method for producing an alcohol-reduced or alcohol-free composition of at least one plant extract, the composition having an alcohol content of less than 10% by volume, in particular less than 5% by volume, preferably less than 2.5% by volume, in particular less than 1.5% by volume or equal to or less than 5,000 ppm (0.5% by volume) or even equal to or more than 0.0%,

comprising the following steps:
(a) providing at least one alcohol-containing plant extract,
(b) adding at least one cyclodextrin or cyclodextrin derivative,
(c) removing the alcohol by means of membrane filtration (permeate),
(d) removing the alcohol-reduced or alcohol-free composition (retentate).

Optionally, the retentate obtained can be used again in step (a), so that a further reduction of the alcohol takes place. The retentate obtained optionally has to be diluted with water.

For the purposes of this invention, “alcohol-free (non-alcoholic)” is understood to mean a content of less than 1.5 percent by volume, preferably equal to or less than 5000 ppm, equal to or more than 0.0 percent by volume of alcohol, in particular ethanol, in a liquid composition. Other auxiliaries and additives may optionally be used, such as preservatives (including potassium sorbate, sorbic acid, sodium benzoate), pH regulators (including citric acid monohydrate), buffers (including sodium gluconate), solubilizers (including glycerine, monopropylene glycol, polyethylene glycol), viscosity enhancers (including polyvinylpyrrolidone, xanthan gum, sodium carboxymethyl cellulose, sodium alginate, maltodextrin, methyl cellulose), solubilizers (including macrogol glycerol hydroxystearate, octenyl starch succinate), viscosity enhancers (including polyvinylpyrrolidone, xanthan gum, sodium carboyxmethyl cellulose, sodium alginate, maltodextrin, methyl cellulose) sweeteners (including sucrose, maltitol, sorbitol, isomalt, sodium saccharin), flavorings, solvents (purified water), dyes, antioxidants (including ascorbic acid), chelating agents (including sodium EDTA), filtration aids (including cross-linked polyvinylpyrrolidone), so that an end product is alcohol-free.

For the purposes of this invention, “cyclodextrin (for short: CD)” is understood to mean a non-reducing cyclic saccharide, also called cyclohexaamylose, consisting of six or more α-1,4-linked D-glucopyranosyl units, which is produced from hydrolyzed starch by the action of cycloglycosyltransferase (CGTase, EC 2.4.1.19). Depending on the number of D-glucopyranosyl units, a distinction is made among alpha, beta, gamma and delta-cyclodextrin, which is suitable for inclusion compounds with one molecule. Also encompassed according to the invention are cyclodextrin derivatives such as alkylcyclodextrins or acylcyclodextrins, for example, methyl-β-cyclodextrin, or hydroxyalkylcyclodextrins, such as 2-hydroxypropyl-β-cyclodextrin, wherein hydroxyl groups of a glucose unit are derivatized.

Van der Waals forces, electrostatic interactions, hydrogen bonds and charge transfer interactions are responsible for the formation of inclusion complexes with CD. Displacement of water from the cavity and reduction of conformational tensions only play a subordinate role.

The binding interactions of α-, β- and γ-CD with, for example, thymol in the crystalline and in the dissolved state are summarized by Ceborska (2018). Bose et al. (2019) spectroscopically examined the interactions of thymol with α- and β-CD and with molecular docking and simulation studies. Accordingly, solvated thymol molecules bind inside α- and β-CD in a molar ratio of 1:1. This increases the size of the thymol molecules.

In the context of this invention, a “plant extract” is understood to mean a multicomponent mixture of natural substances containing more than two natural substances, in particular more than 10 or 100 natural substances, in particular more than 200, 300, 500 or 1,000 natural substances. Plant extracts can be obtained from plant materials, for example, by means of extraction, percolation or maceration. Alcohol-containing solvents, C1-C5 alcohols, and ethanol can be used as extractants. A customary extraction is, for example, an aqueous-ethanolic extraction, in particular a mixture of water/ethanol (50:50, 70:30, 30:70), for example, at 15 to 80 degrees Celsius and normal pressure. The term plant extract also comprises fluid extracts (Extractum fluidum), wherein a liquid drug preparation is provided in which as little extraction liquid as possible is used for the extraction of the drug. This affects the drug to extract ratio (DEV).

According to the invention, the following genera are preferably considered as plant materials, such as Achillea, Aloe, Althaea, Angelica, Arnika, Artemisia, Cannabis, Capsicum, Carum, Caulophyllum, Centaurium, Chelidonium, Cimicifuga, Citrus, Cyclamen, Cynara, Echinacea, Equisetum, Glycyrrhiza, Guaiacum, Hedeara, Humulus, Iberis, Iris, Juglans, Lavandula, Levisticum, Lilium, Matricaria, Melissa, Mentha, Basilicum (Ocimum), Phytolacca, Pimpinella, Primula, Punica, Quercus, Rosmarinus, Rumex, Salix, Salvia, Sambucus, Silybum, Strychnos, Taraxacum, Thymus, Vaccinium, Valeriana, Vebena, Vitex, Vitis.

According to the invention, the following species are preferably considered as plant materials, such as Achillea millefolium, Aloe vera, Althaea officinalis, Angelica archangelica, Arnika montana, Artemisia vulgaris, annua and absinthium, Cannabis indica, Cannabis ruderalis, Cannabis sativa, Capsicum annuum, Carum carvi, Caulophyllum thalictroides, Centaurium, Chelidonium majus, Cimicifuga racemose, Citrus reticulata, Citrus sinensis, Citrus junos, Citrus medica, Citrus maxima, Citrus aurantifolia, Citrus aurantium, Citrus hystrix, Citrus limon, Citrus paradisi, Cistus incanus, Cyclamen purpurascens, Cynara cardunculus, Echinacea purpurea and angust, Equisetum arvense, Glycyrrhiza glabra, Glycyrrhiza inflata, Glycyrrhiza uralensis, Guaiacum officinale und sanctum, Hedeara helix, Humulus lupulus, Iberis amara, Iris, Juglans regia, Lavandula angustifolia, Levisticum officinale, Lilium tigrinum, Matricaria chamomilla, Melissa officinalis, Mentha candensis, Mentha arvensis, Mentha piperita, Ocimum basilicum, Phytolacca americana, Pimpinella anisum, Primula veris, elatior and vulgaris, Punica granatum, Quercus robur, Quercus petraea, Quercus pubescens, Rosmarinus, Rumex cripus, Rumex obtusifolius, Rumex alpinus, Rumex patientia, Rumex acetosa, Rumex acetosella, Rumex thyrsiflorus, Salix purpurea, Salix daphnoides, Salix fragilis, Salvia officinalis, Sambucus nigra, Silybum marianum, Strychnos ignatii Taraxacum officinale, Thymus vulgaris, Vaccinium macrocarpon, Vaccinium myrtillus, Valeriana officinalis, Vebena officinalis, Vitex agnus castus, Vitis vinifera.

It is further advantageous if the concentration of cyclodextrin in the liquid plant extract or fluid extract is preferably 0.1-20% m/m, preferably 3.7-4.2% m/m before the membrane filtration is carried out.

In a further preferred embodiment of the invention of the membrane filtration according to the invention, the pore size of a membrane is approximately 0.0001 to 0.001 μm. Said pore size allows a retention of molecules larger than 100-200 dalton on the membrane. Particularly suitable membrane materials are not limited to polyamides, polysulfones, polyesters, polyethersulfone, polyethylene, cellulose acetate, cellulose and polypropylene. A chemical or physical modification of the materials can influence the selectivity.

The membrane filtration according to the invention, in particular reverse osmosis, is a pressure-operated concentration process. In this case, the liquid in which the concentration of the alcohol is to be reduced is separated from the medium in which the concentration of the alcohol is to be increased by means of a membrane. The membrane is characterized by selectively permeable properties. The process of osmosis is reversed by increasing the hydrostatic pressure, making a mass transfer against the concentration gradient possible. The membrane filtration, in particular reverse osmosis, is driven by the resulting transmembrane pressure. Pressures (pressure difference and/or a pressure gradient and/or hydrostatic pressure difference) of 5-80 bar, preferably 10-60 bar have been found to be particularly suitable with regard to the alcohol and ethanol reduction according to the invention in plant extracts using cyclodextrins. The permeability of the membrane is also controlled by the process temperature. Temperatures between 5-60° C., preferably 25-35° C., have proven to be particularly suitable. Furthermore, it is preferred that the method is preferably run continuously and in a closed system, on the one hand, to minimize the process time and on the other hand, to prevent contamination, cross-contamination and the loss of important ingredients.

In a further embodiment of the invention, an advantageous pre- or post-treatment of the plant extracts can take place, such as disinfection by means of short-term heating, pasteurization, ultra-high-temperature heating, disinfecting filtration, or the like. Preservation with preservatives such as potassium sorbate is also possible.

The invention also relates to an agent, in particular a medicine or food supplement containing an alcohol-free plant extract, the composition having an alcohol content of less than 1.5%, preferably 5,000 ppm, but the alcohol content being greater than zero, and containing at least one cyclodextrin or cyclodextrin derivative, optionally other additives and auxiliaries. It is further preferred that the proportion of cyclodextrin or cyclodextrin derivative is 0.1-5% by weight (calculated on the total mass or total volume of the medicine).

The medicine can preferably be provided in liquid form, in particular in a drug form selected from the group of drops, juice, syrup, infusion, in particular throat spray and disinfectant solutions, nasal spray, liquid preparations for inhalation, rinsing solutions, in particular in combination with physiological and hyperosmolar concentrations of salts or salt mixtures, preferably table salt, in particular sea salt.

### EXAMPLES

The following examples and figures are intended to explain the invention in more detail without, however, restricting it.

### Example 1

1. Production of the Feed Product for Reverse Osmosis

First, two exemplary alcoholic extracts from thyme (thymus) and ivy (Hedeara) were produced. A mixture of 90% ethanol, 85% glycerine and a 10% ammonia solution was used as the extraction agent to produce the thyme fluid extract. 70% ethanol was used for the production of the ivy extract. An ivy fluid extract having 57% (V/V) ethanol was produced by concentrating and combining a portion of the ivy extract.

The two fluid extracts produced, and HP-β-cyclodextrin, were mixed in a ratio of 10:1:1 (fluid extract thyme:fluid extract Hedera:HP-β-cyclodextrin). The present starting product had an ethanol content of 33.091% (V/V) ethanol.

The mixture was diluted with water in a ratio of 1:1 before reverse osmosis was carried out. The feed product prepared in this way for reverse osmosis contained 4.2% HP-β-cyclodextrin and an ethanol content of 16.304% (V/V).

An analytical chromatogram was created from the present mixture in order to determine and characterize the entire range of ingredients of the present feed product. This is required in order to be able to determine in the subsequent experiments whether the range of ingredients in the feed product has changed or not.

### Example 2

2. Ethanol Reduction of the Feed Product Using Cyclodextrin by Membrane Filtration

A laboratory system customary for membrane filtration was used for dealcoholization of the feed product described in Example 1 using cyclodextrin.

To determine the filtration effectiveness, the permeate and retentate produced were analytically characterized in addition to the feed product. Here again, an analytical chromatogram was used for determining all ingredients of the feed product.

The aim of the reverse osmosis was to separate the ethanol contained in the feed by means of membrane filtration, wherein as little as possible of the product ingredients were filtered off into the permeate.

Two membrane filtration tests were carried out with the same feed product for this purpose.

A cleaned membrane filtration system was used in the first experiment. In the second experiment, however, the system was preconditioned with the feed product before the main experiment. Here, the system having built-in membrane was operated with the feed before the main test in order to wet the membrane with product. As a result, all free binding sites of the polymeric membrane are bound with molecules of the feed. The product used for preconditioning was discarded.

The results obtained are summarized in the table below.

TABLE 1
First experiment using a cleaned membrane filtration
system (without preconditioning):

| | | |
|---|---|---|
| | | Theoretical ethanol |
| | | content [% V/V] |
| | | after mixing with |
| | | other auxiliaries to |
| | Ethanol content | form the finished |
| Amount [g] | [% V/V] | medicinal product |

Before reverse osmosis:

| | | | |
|---|---|---|---|
| Starting | 557.4 | 33.091 | 6.56 |
| product | | | |
| Feed product | 1114.7 | 16.304 | 6.52 |

After reverse osmosis:

| | | | |
|---|---|---|---|
| Concentrate | 614.2 | 15.284 | 2.44 |
| (retentate) | | | |
| Permeate | 474.0 | 15.550 | — |

TABLE 2
Second experiment using a preconditioned
membrane filtration system:

| | | | Theoretical ethanol |
|---|---|---|---|
| | | | content [% V/V] |
| | | | after mixing with |
| | | | other auxiliaries to |
| | | Ethanol content | form the finished |
| | Amount [g] | [% V/V] | medicinal product |
| Before reverse osmosis: | | | |
| Starting | 559.9 | 33.091 | 6.56 |
| product | | | |
| Feed product | 1119.8 | 16.236 | 6.49 |
| After reverse osmosis: | | | |
| Concentrate | 794.1 | 16.087 | 2.13 |
| (retentate) | | | |
| Permeate | 298.7 | 15.502 | — |

The concentrates produced, which optimally contain all of the product ingredients, constitute the product. The permeate obtained, which ideally only contains ethanol and water and no product ingredients, is discarded as a waste product. The initial volume was reduced by filtering off the permeate.

Thus, in the two experiments carried out, concentrates could be obtained which now only contain approx. 15-16% (V/V) ethanol, the alcohol content was thus able to be reduced from 33.091% (V/V) to 15-16% (V/V).

By mixing with further auxiliaries and additives, such as preservatives (including potassium sorbate, sorbic acid, sodium benzoate), pH regulators (including citric acid monohydrate), buffers (including sodium gluconate), solubilizers (including glycerine, monopropylene glycol, polyethylene glycol), viscosity enhancers (including polyvinylpyrrolidone, xanthan gum, sodium carboxymethyl cellulose, sodium alginate, maltodextrin, methyl cellulose), solubilizers (including macrogol glycerol hydroxystearate, octenyl starch succinate), viscosity enhancers (including polyvinylpyrrolidone, xanthan gum, sodium carboyxmethyl cellulose, sodium alginate, maltodextrin, methyl cellulose) sweeteners (including sucrose, maltitol, sorbitol, isomalt, saccharin sodium), flavorings, solvents (purified water), dyes, antioxidants (including ascorbic acid), chelating agents (including sodium EDTA), filtration aids (including cross-linked polyvinylpyrrolidone), a medicinal juice (for example, for children) can only contain approx. 2-3% (V/V) ethanol instead of the 5-6% (V/V) ethanol achieved.

The concentrate is continuously diluted with water and further subjected to the reverse osmosis process according to the invention to further reduce the ethanol. The ethanol reduction is carried out up to a residual value of approx. 2% ethanol. An alcohol-free finished medicinal product formulation having less than 5000 ppm of ethanol can be obtained by subsequent mixing with the aforementioned auxiliary substances and additives to form the finished medicinal product.

The evaluation of the analytical chromatograms of the samples from the experiments showed that the ingredient profiles of the feed and concentrate remain unchanged compared to the starting product. No substances whatsoever could be detected in the permeate. In terms of quality, the concentrate produced is identical to the feed and thus to the starting product used.

The reverse osmosis of an extract mixture with the addition of cyclodextrin is thus extremely effective and advantageous with regard to the retention of product ingredients in the retentate.

### Example 3

3. Investigation of the Influence of Cyclodextrin on Membrane Filtration

In a further experiment, the influence of the cyclodextrin in the feed product was specifically investigated when a membrane filtration was carried out. The decisive factor here is whether by adding cyclodextrin, the thymol contained in the feed product can be retained quantitatively more strongly in the retentate and is thus filtered off into the permeate to a lesser extent. Thymol constitutes a very important, pharmacologically relevant ingredient in thyme, but it is one of the low molecular weight compounds, so that it is particularly easy to filter off into the permeate.

Before the start of the experiment, a mixture of fluid extract thyme, fluid extract ivy and HP-β-cyclodextrin was again prepared as the starting product (mixture in a ratio of 10:1:1, as described in Chapter 1).

In order to be able to specifically examine the influence of cyclodextrin on membrane filtration, the same fluid extract mixture was prepared, but without the addition of HP-β-cyclodextrin (mixture of fluid extract thyme with fluid extract ivy in a ratio of 10:1).

Both starting products produced were diluted 1:1 with water so that two identical feed products were produced, one with and one without cyclodextrin.

The feed product having cyclodextrin prepared in this way contained 3.75% HP-β-cyclodextrin and had an ethanol content of 14.631% (V/V).

The feed product without cyclodextrin had a measured ethanol content of 14.073% (V/V).

A laboratory system that is customary for membrane filtration was used to carry out the experiments, with which laboratory system different membranes can be tested simultaneously under the same test conditions. The system was equipped with four different reverse osmosis membranes (membrane A-D) for the experiment.

In the first run, the membrane filtration was carried out with the cyclodextrin-added feed product. For the second run, new A-D membranes were used with the same conditions, the feed product was filtered without cyclodextrin.

A quantitative thymol content determination was carried out for all generated permeates for a sensitive, analytical test monitoring.

TABLE 3
The results of the series of experiments are summarized in the following table:

| | | Feed | Thymol | Thymol | |
|---|---|---|---|---|---|
| | | with/without | content | content | Recovery |
| | | HP-β- | feed | permeate | thymol |
| Name | | cyclodextrin | [mg/100 mg] | [mg/100 mg] | [%] |
| Permeate | Membrane A | With | 0.01927 | 0.00001 | 0.05 |
| Permeate | Membrane A | Without | 0.02261 | 0.00031 | 1.37 |
| Permeate | Membrane B | With | 0.01927 | 0.00001 | 0.05 |
| Permeate | Membrane B | Without | 0.02261 | 0.00035 | 1.55 |
| Permeate | Membrane C | With | 0.01927 | 0.00052 | 2.70 |
| Permeate | Membrane C | Without | 0.02261 | 0.00352 | 15.57 |
| Permeate | Membrane D | With | 0.01927 | 0.00094 | 4.88 |
| Permeate | Membrane D | Without | 0.02261 | 0.00477 | 21.10 |

The evaluation of the analytical results was based on the calculated recoveries of thymol in the permeate. That is, the lower the recovery in the permeate, the lower the transition from thymol to the permeate and the higher the retention of thymol on the retentate side.

The recoveries of thymol in the permeate were significantly lower when using feed with cyclodextrin than in the experiments without cyclodextrin. Thus, the transfer of thymol into the permeate is particularly low with the use of cyclodextrin.

This result could be achieved consistently with all membranes, that is, the effect that cyclodextrin more strongly retains the thymol on the retentate side and thus less thymol is found in the permeate, could be determined regardless of the membrane used. This knowledge is reinforced since the experiment series was carried out with the same starting product, under the same test conditions and with different membranes. That is, system-related fluctuations can be excluded.

### Example 4

4. Carrying Out a Dealcoholization

The aim of a further experiment is to dealcoholize an extract mixture (starting product) mixed with cyclodextrin, wherein the ethanol content of the starting product is reduced from 25% (m/m) to at least ≤3% (m/m). As a result, a finished medicinal product having ≤0.6% (V/V) or ≤0.5% (m/m) ethanol can be produced from this dealcoholized concentrate by mixing it with other auxiliaries.

Although the starting product is subjected to a stronger dealcoholization in this experiment, the range of ingredients from the starting product to the concentrate should remain analytically comparable or identical.

The present experiment was carried out with a larger amount of product and with a pilot plant reverse osmosis system with a membrane module that is customary for membrane filtration.

Two alcoholic extracts from thyme and ivy, which were prepared in the same way as described in Example 1, were used for the experiment. The two fluid extracts were mixed in a ratio of 10:1:1 with HP-β-cyclodextrin. An ethanol content of 32.79% (V/V) could be analyzed in the present starting product. In addition, an analytical chromatogram was created from the starting product in order to determine the entire spectrum of ingredients and thus the original quality.

Preconditioning:

The reverse osmosis system was preconditioned before the dealcoholization process. For this purpose, the system having built-in membrane module with a starting product-water mixture (60.6% starting product, 39.4% water) was run in a cycle in order to wet the polymeric structure of the membrane module with product. The starting product/water mixture used for preconditioning was discarded.

Dealcoholization:

The actual dealcoholization process was started after the preconditioning was carried out. A feed mixture was prepared from 19.35% starting product and 80.65% water for the dealcoholization. The feed product present had an ethanol content of 6.31% (V/V) ethanol and contained 1.6% HP-β-cyclodextrin.

Water was continuously added to the concentrate during the reverse osmosis process in order to continuously reduce the ethanol content in the concentrate. The reverse osmosis was carried out until the concentrate had an ethanol content of ≤3% (m/m) and the amount of concentrate corresponded to the amount of starting product.

The results of the experiment are summarized in Table 4 below:

| | | | Ethanol content after mixing |
|---|---|---|---|
| | Amount | | with further auxiliaries to form |
| | [kg] | Ethanol content | the finished medicinal product |
| Before reverse osmosis: | | | |
| Starting | 11.868 | 32.79% (V/V) // 25.3% (m/m) | 6.12% (V/V) // 4.3% (m/m) |
| product | | | |
| Feed product | 61.333 | 6.31% (V/V) // 5.0% (m/m) | — |
| After reverse osmosis: | | | |
| Concentrate | 11.162 | 3.81% (V/V) // 2.8% (m/m) | 0.62% (V/V) // 0.4% (m/m) |
| (retentate) | | | |
| Permeate | 103.117 | 4.68% (V/V) // 3.7% (m/m) | — |

The present results show that the dealcoholization of the starting product was successful and an ethanol content of ≤3% (m/m) could be achieved in the concentrate. The finished medicinal product produced from the concentrate had an ethanol content of 0.4% (m/m) ethanol.

In the area of herbal active ingredients, the ethanolic aqueous extraction brings a significantly larger range of ingredients into solution than a purely aqueous extraction. This spectrum of active ingredients and ingredients must be preserved through gentle, subsequent dealcoholization, thus preserving the elution power of the ethanol. The analytical investigations of the samples from the experiments therefore focused on examining the changes in the overall composition of the complex mixtures due to reverse osmosis in connection with cyclodextrin.

As a summary of the findings of the present model experiment with thyme and ivy, it can again be confirmed in this experiment that the dealcoholization of the starting product (starting mixture consisting of thyme fluid extract, ivy leaf extract and cyclodextrin) was successful. The initial ethanol content of 25% (m/m) could be reduced to approx. 2.8% (m/m).

The other components in the starting product are virtually unchanged in terms of quality and quantity at the end of the process.

TABLE 5
Qualitative tests - comparison of ingredient
profiles before and after dealcoholization

| | | |
|---|---|---|
| | | End |
| Test parameters | Starting product | product(s) |

| | |
|---|---|
| DC fingerprint on saponins | Fingerprint indistinguishable |
| DC fingerprint on flavonoids | Fingerprint indistinguishable |
| GC fingerprint (direct | Hardly any differences in the |
| injection) | RT ranges 6-43 min |
| HPLC MS chromatograms | Visually very good agreement |
| | of 896 signals |

TABLE 6
Quantitative results - recovery of individual
compounds after dealcoholization

| | Recovery in the |
|---|---|
| | concentrate in |
| | relation to the |
| Quantitative parameters | starting product |
| Thymol | 99.8% |
| Linalool | 102.9% |
| Terpinene-4-ol | 98.1% |
| Carvacrol | 99.1% |
| Eucalyptol | 103.3% |
| Camphor | 98.4% |
| Carophylline oxide | 111.8% |
| Eugenol | 102.0% |
| Hederacoside C | 97.0% |
| Agreement GC (sum of the peaks | 98.3% |
| according to Ph. Eur. 1374 > LOQ) | |

Fingerprints:

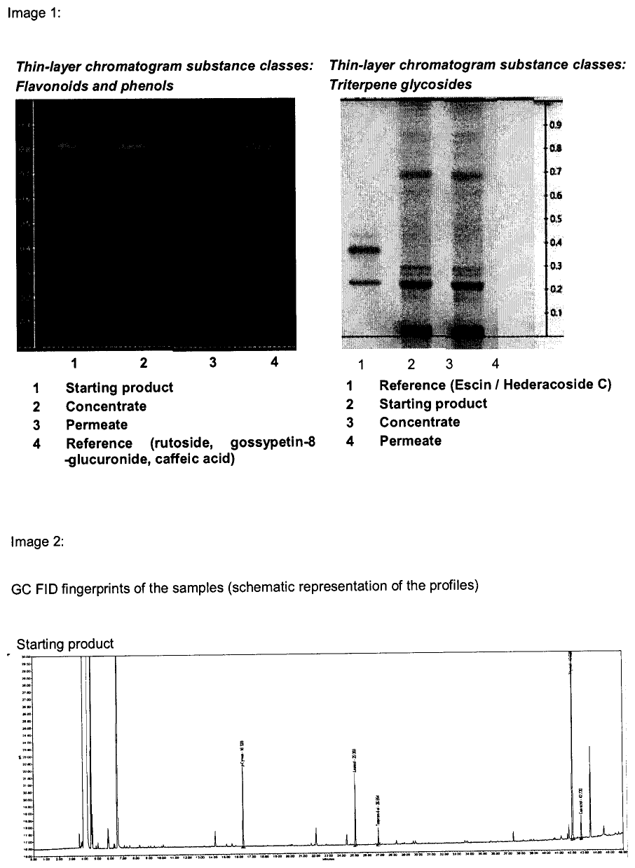

In addition to the quantitative methods, qualitative fingerprint methods were also used to compare profiles. The efficiency of the technology is particularly evident in the substance class of flavonoids and saponins. The cyclodextrin-mediated complexation, which is also described for these substance classes, prevents these substances from being lost during dealcoholization on the opposite side of the membrane into the permeate (see FIGS. 1A and 1B).

FIGS. 1A and 1B show thin-layer chromatograms (TLC fingerprint).

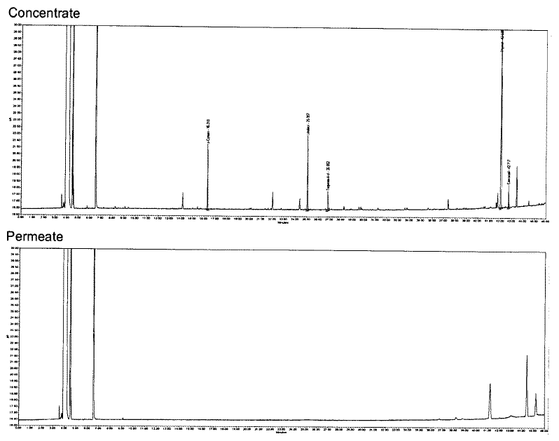

The method with direct injection, based on the Ph. Eur. Testing for essential oils of thyme is usually evaluated in the retention time range of 6-43 minutes. The chromatograms presented in FIGS. 2A, 2B and 2C show the comparison of the total chromatograms for the samples starting product, concentrate and permeate.

In the evaluation range defined above, in which the volatile components of the samples are recorded (among others, essential oil components), the chromatograms of the starting product and concentrate are practically identical. No signals can be seen in the permeate in this range.

FIGS. 2A, 2B and 2C show GC FID fingerprints.

### LITERATURE

Bose, A., Sengupta, P., Pal, U., Senapati, S., Ahsan, M., Roy, S., Das, U., Sen, K.: Encapsulation of thymol in cyclodextrin nano-cavities: a multi spectroscopic and theoretical study. Spectrochim. Acta Part A: Mol. Biomol. Spectr. 2019, 208, 339-348.
Brewster, M. E., Loftsson, T.: Cyclodextrins as pharmaceutical solubilizers. Adv. Drug Deliv. Rev. 2007, 59, 645-666.
Ceborska, M.: Structural investigation of solid state host/guest complexes of native cyclodextrins with monoterpenes and their simple derivatives. J. Mol. Struct. 2018, 1165, 62-70.
Davis, M. E., Brewster, M. E.: Cyclodextrin-based pharmaceutics: past, present and future. Nature Reviews 2004, 3, 1023-1035.
de Oliveira-Filho, e Silva, A. R. A., de Azevedo Moreira, R., Nogueira, N. A. P.: Biological activities and pharmacological applications of cyclodextrins complexed with essential oils and their volatile components: a systematic review. Curr. Pharm. Design 2018, 24 (33), 3951-3963.
Lima, P. S. S., Lucchese, A. M., Aradjo-Filho, H. G., Menezes, P. P., Araujo, A. A. S., Quintans-Junior, L. J., Quintans, J. S. S.: Inclusion of terpenes in cyclodextrins: preparation, characterization and pharmacological approaches. Carbohydr. Polym. 2016, 151, 965-987.
Loftsson, T., Duchene, D.: Cyclodextrins and their pharmaceutical applications. Int. J. Pharmaceutics 2007, 329, 1-11.
Marques, H. M. C.: A review on cyclodextrin encapsulation of essential oils and volatiles. Flavour Fragr. J. 2010, 25, 313-326.
Pivetta, T. P., Simoes, S., Araujo, M M., Carvalho, T., Arruda, C., Marcato, P. D.: Development of nanoparticles from natural lipids for topical delivery of thymol: investigation of its anti-inflammatory properties. Colloids Surf. B Biointerfaces 2018, 164, 281-290.
Szejtli, J.: Medicinal applications of cyclodextrins. Med. Res. Rev. 1994, 14 (3), 353-386.
Wadhwa, G., Kumar, S., Chhabra, L., Mahant, S., Rao, R.: Essential oil-cyclodextrin complexes: An updated review. J. Incl. Phenom. Macrocycl. Chem. 2017, 89, 39-58.

1.-14. (canceled)

15. A method for producing a composition of at least one plant extract, the composition having an alcohol content of less than 10 Vol. % comprising the following steps:
(a) providing at least one alcohol-containing plant extract,
(b) adding at least one cyclodextrin or cyclodextrin derivative,
(c) removing the alcohol by means of membrane filtration (permeate) and
(d) removing the alcohol-reduced composition (retentate).

16. The method for producing a composition of at least one plant extract according to claim 15, wherein the composition has an alcohol content of less than 5 Vol. %.

17. The method for producing a composition of at least one plant extract according to claim 15, wherein the composition has an alcohol content of less than 1.5 Vol. % wherein an alcohol-free composition is removed in step (d).

18. The method for producing a composition of at least one plant extract according to claim 15, wherein the composition has an alcohol content of less than 5,000 ppm, wherein an alcohol-free composition is removed in step (d).

19. The method for producing a composition of at least one plant extract according to claim 15, wherein the retentate from step (d) is used in step (a) and, is optionally diluted with water.

20. The method for producing a composition of at least one plant extract according to claim 15, wherein the membrane filtration in step (c) is an ultrafiltration, nanofiltration or reverse osmosis.

21. The method for producing a composition of at least one plant extract according to claim 15, wherein the cyclodextrin or cyclodextrin derivative in step (b) is selected from the group consisting of alpha, beta, gamma and delta-cyclodextrin, alkylcyclodextrins, acylcyclodextrins, methyl-β-cyclodextrin, hydroxyalkyl cyclodextrins, and 2-hydroxypropyl-β-cyclodextrin.

22. The method for producing a composition of at least one plant extract according to claim 15, wherein the concentration of cyclodextrin in the liquid plant extract or fluid extract before the membrane filtration is carried out is 0.1-20% m/m.

23. The method for producing a composition of at least one plant extract according to claim 15, wherein the concentration of cyclodextrin in the liquid plant extract or fluid extract before the membrane filtration is carried out is 3.7-4.2% m/m.

24. The method for producing a composition of at least one plant extract according to claim 15, wherein the membrane filtration is carried out with a pressure difference and/or a pressure gradient and/or hydrostatic pressure difference in the range of 5-80 bar.

25. The method for producing a composition of at least one plant extract according to claim 15, wherein the membrane filtration is carried out at a process temperature between 5-60° C.

26. The method for producing a composition of at least one plant extract according to claim 15, wherein the membrane filtration is by reverse osmosis, and is carried out with a pressure difference and/or a pressure gradient and/or hydrostatic pressure difference in the range of 5-80 bar and at a process temperature between 5-60° C.

27. The method for producing a composition of at least one plant extract according to claim 15, wherein the at least one plant extract is selected from a genus from the group consisting of Achillea, Aloe, Althaea, Angelica, Arnika, Artemisia, Cannabis, Capsicum, Carum, Caulophyllum, Centaurium, Chelidonium, Cimicifuga, Citrus, Cyclamen, Cynara, Echinacea, Equisetum, Glycyrrhiza, Guaiacum, Hedeara, Humulus, Iberis, Iris, Juglans, Lavandula, Levisticum, Lilium, Matricaria, Melissa, Mentha, Basilicum (Ocimum), Phytolacca, Pimpinella, Primula, Punica, Quercus, Rosmarinus, Rumex, Salix, Salvia, Sambucus, Silybum, Strychnos, Taraxacum, Thymus, Vaccinium, Valeriana, Vebena, Vitex and Vitis.

28. The method for producing a composition of at least one plant extract according to claim 15, wherein the at least one plant extract is selected from a genus from the group consisting of Thymus and Hedeara.

29. A medicine or food supplement containing an alcohol-free plant extract, the composition having an ethanol content of less than 1.5 Vol. %, but the ethanol content being greater than 0.0 Vol. %, and containing at least one cyclodextrin or cyclodextrin derivative, optionally further additives and auxiliaries.

30. The medicine or food supplement according to claim 29, wherein the composition having an ethanol content of less than 5,000 ppm, but the ethanol content being greater than 0.0 Vol. %, and containing at least one cyclodextrin or cyclodextrin derivative, optionally further additives and auxiliaries.

31. A medicine or food supplement containing an alcohol-free plant extract, the composition having an ethanol content of less than 1.5 Vol. %, but the ethanol content being greater than 0.0 Vol. %, and containing at least one cyclodextrin or cyclodextrin derivative, optionally further additives and auxiliaries obtained by the method according to claim 15.

32. The medicine or food supplement containing an alcohol-free plant extract according to claim 29, wherein the proportion of cyclodextrin or cyclodextrin derivative is 0.1-5% by weight.

33. The medicine or food supplement containing an alcohol-free plant extract according to claim 29, in liquid form selected from the group of drops, juice, syrup, infusion, throat spray and disinfectant solutions, nasal sprays, liquid preparations for inhalation, rinsing solutions, and in combination with physiological and hyperosmolar concentrations of salts or salt mixtures.

34. The medicine or food supplement containing an alcohol-free plant extract according to claim 29, in a medicinal form selected from the group of drops, juice, syrup, infusion, throat spray and disinfectant solutions, nasal sprays, liquid preparations for inhalation, rinsing solutions, and in in combination with physiological and hyperosmolar concentrations of table salt.
